(19) United States
(12) Patent Application Publication
(10) Pub. No.: US 2023/0086433 A1
(43) Pub. Date: Mar. 23, 2023

(54) METHODS FOR THE PREPARATION OF ETHYL 3-BROMO-1-(3-CHLOROPYRIDIN-2-YL)-1H-PYRAZOLE-5-CARBOXYLATE

(71) Applicants: FMC Corporation, Philadelphia, PA (US); FMC Agro Singapore Pte. Ltd., Singapore (SG)

(72) Inventors: Chunyan HUO, Shanghai (CN); Jingyi MA, Shanghai (CN); Shuoen WANG, Shanghai (CN)

(21) Appl. No.: 17/791,353

(22) PCT Filed: Jan. 8, 2021
(86) PCT No.: PCT/US2021/012807

§ 371 Date: Jul. 7, 2022

Related U.S. Application Data
(60) Provisional application No. 62/958,404, filed on Jan. 8, 2020.

(51) Int. Cl. C07D 401/04

(52) U.S. Cl. CPC C07D 401/04

(57) ABSTRACT

Described herein are novel methods of synthesizing Ethyl 3-bromo-1-(3-chloropyridin-2-yl)-1H-pyrazole-5-carboxylate.

### CROSS-REFERENCE TO RELATED APPLICATIONS

This application claims the benefit of U.S. Provisional Application No. 62/958,404 filed Jan. 8, 2020.

### FIELD OF INVENTION

This disclosure is directed to novel methods of synthesizing ethyl 3-bromo-1-(3-chloropyridin-2-yl)-1H-pyrazole-5-carboxylate. Compounds prepared by the methods disclosed herein are useful for preparation of certain anthranilamide compounds that are of interest as insecticides, such as, for example, the insecticides chlorantraniliprole and cyantraniliprole.

### BACKGROUND

The present disclosure provides novel methods useful for preparing ethyl 3-bromo-1-(3-chloropyridin-2-yl)-1H-pyrazole-5-carboxylate and derivatives thereof. Compared to conventional processes using potassium persulfate as an oxidant, bromine as oxidant improves the yield moderately (93-95% versus 80-88%). Furthermore, compared to difficult handling conditions and possible equipment corrosion in conventional bromine heating processes, the photo-catalyzed process described herein utilizes significantly milder reaction conditions (25-65° C./normal pressures versus 125-130° C./vacuum) and reaction times (30-45 minutes versus about 8 hours). Overall, the benefits of the methods of the present disclosure compared to previous methods are numerous and include improved overall yield, reduced cost, milder reaction conditions, and shorter reaction times.

### BRIEF DESCRIPTION

In one aspect, provided herein is a method of preparing a compound of Formula (II)

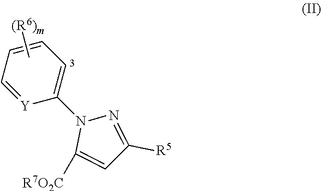

wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;

### Y is N or CR⁸;

R⁸ is H or R⁹, wherein R⁹ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; and
m is 0, 1, 2, or 3, with the proviso that when Y is CH then m is at least 1, the method comprising:

I) forming a mixture comprising
A) a compound of Formula (I)

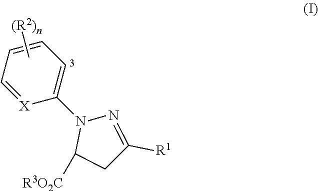

wherein R¹ is halogen;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) an organic solvent; and
C) an inorganic base;

II) optionally heating the mixture;

III) irradiating the mixture; and

IV) adding an oxidizing agent to the mixture.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “alkyl”, used either alone or in compound words such as “alkylthio” or “haloalkyl” includes straight-chain or branched alkyl, such as methyl, ethyl, n-propyl, i-propyl, or the different butyl, pentyl or hexyl isomers.

The term “alkenyl” can include straight-chain or branched alkenes such as 1-propenyl, 2-propenyl, and the different butenyl, pentenyl and hexenyl isomers. “Alkenyl” also includes polyenes such as 1,2-propadienyl and 2,4-hexadienyl.

The term “alkynyl” includes straight-chain or branched alkynes such as 1-propynyl, 2-propynyl and the different butynyl, pentynyl and hexynyl isomers. “Alkynyl” can also include moieties comprised of multiple triple bonds such as 2,5-hexadiynyl.

The term “alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂.

The term “alkylthio” includes branched or straight-chain alkylthio moieties such as methylthio, ethylthio, and the different propylthio, butylthio, pentylthio and hexylthio isomers.

The term “cycloalkyl” includes, for example, cyclopropyl, cyclobutyl, cyclopentyl and cyclohexyl. “Cycloalkylalkyl” indicates an alkyl group substituted with a cycloalky group and includes, for example, cyclopropylmethyl, cyclobutylethyl, cyclopentylpropyl and cyclohexylmethyl.

The term “cycloalkylamino” means the amino nitrogen atom is attached to a cycloalkyl radical and a hydrogen atom and includes groups such as cyclopropylamino, cyclobutylamino, cyclopentylamino and cyclohexylamino. “(Alkyl)cycloalkylamino” means a cycloalkylamino group where the hydrogen atom is replaced by an alkyl radical; examples include groups such as (alkyl)cyclopropylamino, (alkyl)cyclobutylamino, (alkyl)cyclopentylamino and (alkyl)cyclohexylamino.

The term “aryl” refers to an aromatic ring or ring system or a heteroaromatic ring or ring system, each ring or ring system optionally substituted. The term “aromatic ring system” denotes fully unsaturated carbocycles and heterocycles in which at least one ring of a polycyclic ring system is aromatic. Aromatic indicates that each of ring atoms is essentially in the same plane and has a p-orbital perpendicular to the ring plane, and in which (4n+2) π electrons, when n is 0 or a positive integer, are associated with the ring to comply with Hückel's rule. The term “aromatic carbocyclic ring system” includes fully aromatic carbocycles and carbocycles in which at least one ring of a polycyclic ring system is aromatic (e.g. phenyl and naphthyl). The term “heteroaromatic ring or ring system” includes fully aromatic heterocycles and heterocycles in which at least one ring of a polycyclic ring system is aromatic and in which at least one ring atom is not carbon and can contain 1 to 4 heteroatoms independently selected from the group consisting of nitrogen, oxygen and sulfur, provided that each heteroaromatic ring contains no more than 4 nitrogens, no more than 2 oxygens and no more than 2 sulfurs (where aromatic indicates that the Hückel rule is satisfied). The heterocyclic ring systems can be attached through any available carbon or nitrogen by replacement of a hydrogen on said carbon or nitrogen. More specifically, the term “aryl” refers to the moiety

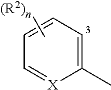

wherein R² and n are defined as above and the “3” indicates the 3-position for substituents on the moiety.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkynyl”, “haloalkoxy”, and the like, are defined analogously to the term “haloalkyl”. Examples of “haloalkenyl” include (Cl)₂C═CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, HCF₂CH₂CH₂O and CF₃CH₂O.

The terms “alkylaminocarbonyl” and “dialkylaminocarbonyl” include, for example, CH₃NHC(═O), CH₃CH₂NHC(═O) and (CH₃)₂NC(═O).

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl. In the above recitations, when a compound of Formula (I) contains a heteroaromatic ring, all substituents are attached to this ring through any available carbon or nitrogen by replacement of a hydrogen on said carbon or nitrogen.

When a group contains a substituent which can be hydrogen, for example R⁴, then, when this substituent is taken as hydrogen, it is recognized that this is equivalent to said group being unsubstituted.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula (II)

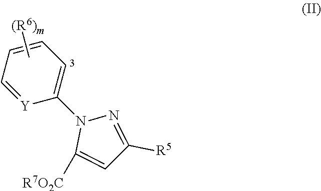

wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;

### Y is N or CR⁸;

R^{s} is H or R⁹, wherein R⁹ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; and
m is 0, 1, 2, or 3, with the proviso that when Y is CH then m is at least 1, the method comprising:

I) forming a mixture comprising
A) a compound of Formula (I)

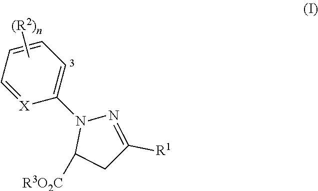

wherein R¹ is halogen;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) an organic solvent; and
C) an inorganic base;

II) optionally heating the mixture;

III) irradiating the mixture; and

IV) adding an oxidizing agent to the mixture.

Embodiment 2. The method of embodiment 1 wherein m is 1, 2, or 3.

Embodiment 3. The method of embodiment 1 or 2 wherein R⁵ is Cl or Br.

Embodiment 4. The method of any one of embodiments 1-3 wherein R⁶ is independently Cl or Br.

Embodiment 5. The method of embodiment 4 wherein one R⁶ is at the 3-position.

Embodiment 6. The method of any one of embodiments 1-5 wherein R⁷ is C₁-C₄ alkyl.

Embodiment 7. The method of any one of embodiment 1-6 wherein Y is N.

Embodiment 8. The method of any one of embodiments 1-7 wherein the compound of Formula (II) is ethyl 3-bromo-1-(3-chloro-2-pyridinyl)-1H-pyrazole-5-carboxylate, having the following structure:

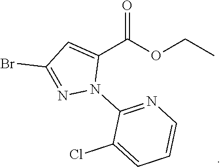 [SMILES: CCOC(=O)c1cc(Br)nn1-c1ncccc1Cl]

Embodiment 9. The method of any one of embodiments 1-8, further comprising isolating the compound of Formula (II).

Embodiment 10. The method of any one of embodiments 1-9 wherein n is 1, 2, or 3.

Embodiment 11. The method of any one of embodiments 1-10 wherein R¹ is Cl or Br.

Embodiment 12. The method of any one of embodiments 1-11 wherein R² is independently Cl or Br.

Embodiment 13. The method of embodiment 12 wherein one R² is at the 3-position.

Embodiment 14. The method of any one of embodiments 1-13 wherein R³ is C₁-C₄ alkyl.

Embodiment 15. The method of any one of embodiment 1-14 wherein X is N.

Embodiment 16. The method of any one of embodiments 1-15 wherein the compound of Formula (I) is ethyl 3-bromo-1-(3-chloropyridin-2-yl)-4,5-dihydro-1H-pyrazole-5-carboxylate, having the following structure:

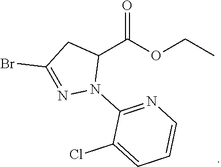 [SMILES: CCOC(=O)C1CC(Br)=NN1c1ncccc1Cl]

Embodiment 17. The method of any one of embodiments 1-16 wherein the organic solvent is selected from acetonitrile, N,N-dimethylformamide, dimethylacetamide, chloroform, acetone, propionitrile, chlorobutane, chlorobenzene, tetrachloromethane, dichlorobenzene, dichloromethane, 1,2-dichloroethane, and combinations thereof.

Embodiment 18. The method of any one of embodiments 1-17 wherein the inorganic base is selected from sodium bicarbonate, potassium bicarbonate, calcium bicarbonate, sodium carbonate, potassium carbonate, ammonium carbonate, ammonium bicarbonate, trisodium phosphate, tripotassium phosphate, caesium carbonate, triethylamine, pyridine, N-methylimidazole, potassium hydrogen phosphate, sodium hydrogen phosphate, sodium hydroxide, potassium hydroxide, and combinations thereof.

Embodiment 19. The method of any one of embodiments 1-18 wherein the inorganic base is in the form of a solid or an aqueous solution.

Embodiment 20. The method of any one of embodiments 1-19 wherein the mixture is a two-phase mixture.

Embodiment 21. The method of any one of embodiments 1-20 wherein the oxidizing agent is selected from bromine, H₂O₂, K₂S₂O₈, and combinations thereof.

Embodiment 22. The method of any one of embodiments 1-21 wherein the method step of adding an oxidizing agent to the mixture comprises continuously adding the oxidizing agent.

Embodiment 23. The method of any one of embodiments 1-22 wherein the method step of adding an oxidizing agent to the mixture comprises dropwise addition of the oxidizing agent.

Embodiment 24. The method of any one of embodiments 1-23 wherein the method step of adding an oxidizing agent to the mixture occurs over a period of time in the range of about 1 minute to about 1 hour.

Embodiment 25. The method of any one of embodiments 1-24, wherein the method step of irradiating the mixture occurs at a temperature in the range of about 20° C. to about 70° C.

Embodiment 26. The method of any one of embodiments 1-25, wherein the method step of irradiating the mixture occurs at a temperature in the range of about 20° C. to about 45° C.

Embodiment 27. The method of any one of embodiments 1-26, wherein the method step of heating the mixture increases the temperature of the mixture to a temperature in the range of about 50° C. to about 82° C.

Embodiment 28. The method of any one of embodiments 1-27, wherein the method step of irradiating the mixture is achieved with a light source selected from a UV lamp, a metal halide lamp, a visible light lamp, and combinations thereof.

Embodiment 29. The method of any one of embodiments 1-28, wherein the method step of irradiating the mixture occurs in the presence of visible light.

Embodiment 30. The method of any one of embodiments 1-29, wherein the method step of irradiating the mixture occurs at a power in the range of about 50 W to about 300 W.

Embodiment 31. The method of any one of embodiments 1-30 wherein at least one method step further comprises stirring the mixture.

Embodiment 32. The method of any one of embodiments 1-31 wherein at least one method step occurs at ambient pressure.

Embodiment 33. The method of any one of embodiments 1-32 wherein the reaction occurs in a batch reactor, a batch-rope reactor, or a flow reactor.

Embodiment 34. The method of any one of embodiments 1-33 wherein the compound of Formula (I) is present in a purity less than about 99%.

Embodiment 35. The method of any one of embodiments 1-34 wherein the compound of Formula (I) is present in a purity less than about 98%.

Embodiment 36. The method of any one of embodiments 1-35 wherein the compound of Formula (I) is present in a purity less than about 97%.

Embodiment 37. The method of any one of embodiments 1-36 wherein the compound of Formula (I) is present in a purity less than about 95%.

In one aspect, Ethyl 3-bromo-1-(3-chloropyridin-2-yl)-1H-pyrazole-5-carboxylate is prepared according to a method represented by Scheme 1.

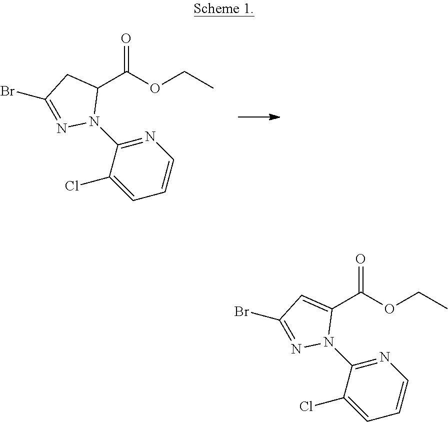 [SMILES: CCOC(=O)C1CC(Br)=NN1c1ncccc1Cl ; CCOC(=O)c1cc(Br)nn1-c1ncccc1Cl]

In one aspect, a compound of Formula II is prepared according to a method represented by Scheme 2. The R groups, X, Y, n, and m are as defined anywhere in this disclosure.

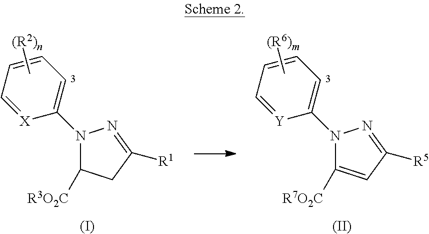

This aspect includes forming a mixture comprising a compound of Formula I, an organic solvent, and an inorganic base, optionally heating the mixture, irradiating the mixture, and adding an oxidizing agent to the mixture. In one embodiment, the reaction of the mixture is complete after completion of the addition of the oxidizing agent. In another embodiment, the reaction of the mixture is complete after the irradiation source is removed.

In one embodiment, the mixture is a two-phase mixture.

In one embodiment, the organic solvent is selected from acetonitrile, N,N-dimethylformamide, dimethylacetamide, chloroform, acetone, propionitrile, chlorobutane, chlorobenzene, tetrachloromethane, dichlorobenzene, dichloromethane, 1,2-dichloroethane, and combinations thereof. In another embodiment the organic solvent is selected from chlorobutane, chlorobenzene, and combinations thereof. In another embodiment, the organic solvent is chlorobutane.

In one embodiment, the inorganic base is selected from sodium bicarbonate, potassium bicarbonate, calcium bicarbonate, sodium carbonate, potassium carbonate, ammonium carbonate, ammonium bicarbonate, trisodium phosphate, tripotassium phosphate, caesium carbonate, triethylamine, pyridine, N-methylimidazole, potassium hydrogen phosphate, sodium hydrogen phosphate, sodium hydroxide, potassium hydroxide, and combinations thereof. In another embodiment, the inorganic base is selected from potassium bicarbonate, pyridine, and combinations thereof. In another embodiment, the inorganic base is potassium bicarbonate.

In one embodiment, the inorganic base is in the form of a solid or an aqueous solution.

In one embodiment, the oxidizing agent is selected from bromine, H₂O₂, K₂S₂O₈, and combinations thereof. In another embodiment, the oxidizing agent is bromine.

In one embodiment, the method step of adding an oxidizing agent to the mixture comprises continuously adding the oxidizing agent. In another embodiment, the method step of adding an oxidizing agent to the mixture comprises dropwise addition of the oxidizing agent. In another embodiment, the method step of adding an oxidizing agent to the mixture occurs over a period of time in the range of about 1 minute to about 1 hour.

In one embodiment, the method step of irradiating the mixture occurs at a temperature in the range of about 20° C. to about 70° C. In another embodiment, the method step of irradiating the mixture occurs at a temperature in the range of about 20° C. to about 45° C.

In one embodiment, the method step of irradiating the mixture is achieved with a light source selected from a UV lamp, a metal halide lamp, a visible light lamp, and combinations thereof. In one embodiment, the method step of irradiating the mixture occurs in the presence of visible light. In one embodiment, the method step of irradiating the mixture occurs at a wavelength in the range of about 350 nm to about 850 nm. In one embodiment, the method step of irradiating the mixture occurs at a power in the range of about 50 W to about 300 W. In another embodiment, the method step of irradiating the mixture occurs at a power in the range of about 75 W to about 250 W.

In one embodiment, the method step of heating the mixture increases the temperature of the mixture to a temperature in the range of about 50° C. to about 82° C.

In one embodiment, at least one method step further comprises stirring the mixture. In one embodiment, at least one method step occurs at ambient pressure.

In one embodiment, the reaction occurs in a batch reactor, a batch-rope reactor, or a flow reactor.

In one embodiment, the compound of Formula (I) is present in a purity less than about 99%. In another embodiment, the compound of Formula (I) is present in a purity less than about 98%. In another embodiment, the compound of Formula (I) is present in a purity less than about 97%. In another embodiment, the compound of Formula (I) is present in a purity less than about 95%.

In one embodiment, the compound of Formula (I) is in a crude reaction mixture with a bromine radical inhibitor.

Conditions which favor the formation of bromine radicals, such as high temperature or the presence of light, favor the formation of ethyl 3-bromo-1-(3-chloropyridin-2-yl)-1H-pyrazole-5-carboxylate. Compared with thermal initiation, bromine initiation can be induced with strong light at moderate temperature.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. Bromine Oxidation Under UV Light

In a jacketed vessel, 25 g ethyl 3-bromo-1-(3-chloropyridin-2-yl)-4,5-dihydro-1H-pyrazole-5-carboxylate in 500 g 1-chlorobutane is kept at ambient temperature. 15 g potassium bicarbonate is added in one portion, and the mixture is kept stirring by a mechanical agitator. A specialized quartz cap with an inward tube is placed at the top of the vessel to seal, and a UV lamp (254 nm, 30 W) is inserted into the tube ready to irritate. The reaction mixture is then heated to 60° C. As soon as the lamp is turned on, a dilution of 14.4 g bromine in 20 g 1-chlorobutane is added dropwise over 45 minutes. After completion of the addition, the mixture components are identified by HPLC, and the area percent of ethyl 3-bromo-1-(3-chloropyridin-2-yl)-1H-pyrazole-5-carboxylate is 77%.

15-25% impurities of ethyl 3-bromo-1-(5-bromo-3-chloropyridin-2-yl)-4,5-dihydro-1H-pyrazole-5-carboxylate and ethyl 3-bromo-1-(5-bromo-3-chloropyridin-2-yl)-1H-pyrazole-5-carboxylate were observed in the product. These impurities could be decreased to about 10% when the UV lamp power was increased from 30 W to over 100 W.

### Example 2. Bromine Oxidation Under Visible Light

In a jacketed vessel, 25 g ethyl 3-bromo-1-(3-chloropyridin-2-yl)-4,5-dihydro-1H-pyrazole-5-carboxylate in 500 g 1-chlorobutane is kept at ambient temperature. 15 g potassium bicarbonate in 100 g water is added in one portion, and the mixture is kept stirring by a mechanical agitator. A specialized quartz cap with an inward tube is placed at the top of the vessel to seal, and a metal-halide lamp (100 W) is inserted into the tube ready to irritate. The reaction mixture is then heated to 65° C. As soon as the lamp is turned on, a dilution of 14.4 g bromine in 20 g 1-chlorobutane is added dropwise over 45 minutes. The reaction temperature can be observed rising to 70° C. while adding bromine. After completion of the addition, the reaction is ceased by shutting down the lamp, and cooling to room temperature. The two-phase mixture is separated, and the organic phase is collected without further extraction from the aqueous phase. The solvent is then removed under reduced pressure, and the resulting crude product is recrystallized using 75 g ethanol and 25 g water. Afterwards, the product is collected by filtration, and the yield of ethyl 3-bromo-1-(3-chloropyridin-2-yl)-1H-pyrazole-5-carboxylate is 93%.

The reaction completed at the exact moment all the oxidant was added. Major impurities in the crude product were identified as trace amounts of ethyl 3-bromo-1-(5-bromo-3-chloropyridin-2-yl)-4,5-dihydro-1H-pyrazole-5-carboxylate and ethyl 3-bromo-1-(5-bromo-3-chloropyridin-2-yl)-1H-pyrazole-5-carboxylate impurities.

### Example 3. Bromine Oxidation in a Batch-Rope Reactor

In a batch-rope model, a solution of crude ethyl 3-bromo-1-(3-chloropyridin-2-yl)-4,5-dihydro-1H-pyrazole-5-carboxylate in chlorobutane (2-15%) and an aqueous solution of potassium bicarbonate are mixed in the flask with strong stirring. The mixture is then injected by a peristaltic pump into an irradiation pool which is equipped with a metal-halide lamp (100 W), and then returned into the flask as a closed loop circuit. A solution of bromine in chlorobutane is pumped into the irradiation pool by portions with the speed controlled sufficient mixing over 20-40° C. The reaction is completed after injection of bromine with 98% conversion. The isolation of ethyl 3-bromo-1-(3-chloropyridin-2-yl)-1H-pyrazole-5-carboxylate can reach 92-95%.

One benefit of the batch-rope model is that the illumination unit which was positioned on the inside of the batch reactor in Examples 1 and 2 is isolated from the reactor and mounted outside the reactor to reduce shadows. The reaction completed at the exact moment all the oxidant was added. Major impurities in the crude product were identified as trace amounts of ethyl 3-bromo-1-(5-bromo-3-chloropyridin-2-yl)-4,5-dihydro-1H-pyrazole-5-carboxylate and ethyl 3-bromo-1-(5-bromo-3-chloropyridin-2-yl)-1H-pyrazole-5-carboxylate impurities.

### Example 4. Bromine Oxidation in a Flow Reactor

A specialized reactor is provided, characterized by an irradiation tube in the center, which is surrounded by a spiral tube and bath jacket. The temperature of the cycling water bath is set to 40° C. A solution of crude ethyl 3-bromo-1-(3-chloropyridin-2-yl)-4,5-dihydro-1H-pyrazole-5-carboxylate in chlorobutane (2-15%) is injected by a peristaltic pump and mixed with another aqueous solution flow of potassium bicarbonate ahead of the reactor. These two peristaltic pumps are adjusted to proper injection rates to achieve a good phase mixture. A solution of bromine in chlorobutane is injected by a third peristaltic pump, to be mixed with the above-mentioned two-phase mixture at the exact inlet of the reactor, and the metal-halide lamp (100 W) is turned on. The resulting reaction output is collected by a flask at the outlet, and the components are identified by HPLC. The area percent of ethyl 3-bromo-1-(3-chloropyridin-2-yl)-1H-pyrazole-5-carboxylate is greater than 96%, and the finally obtained product is present in a yield of 90-95%.

Major impurities in the crude product were identified as trace amounts of ethyl 3-bromo-1-(5-bromo-3-chloropyridin-2-yl)-4,5-dihydro-1H-pyrazole-5-carboxylate and ethyl 3-bromo-1-(5-bromo-3-chloropyridin-2-yl)-1H-pyrazole-5-carboxylate impurities. These impurities could be controlled under 1.0% argon. Surprisingly, crude ethyl 3-bromo-1-(3-chloropyridin-2-yl)-4,5-dihydro-1H-pyrazole-5-carboxylate produces higher conversation compared to pure ethyl 3-bromo-1-(3-chloropyridin-2-yl)-4,5-dihydro-1H-pyrazole-5-carboxylate.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula (II) 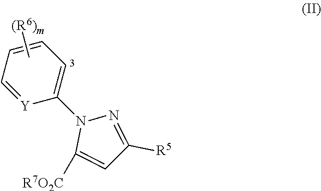
wherein R⁵ is halogen;
each R⁶ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R⁷ is H or C₁-C₄ alkyl;
Y is N or CR⁸;
R^{s} is H or R⁹, wherein R⁹ is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; and
m is 0, 1, 2, or 3, with the proviso that when Y is CH then m is at least 1, the method comprising:
I) forming a mixture comprising
A) a compound of Formula (I) 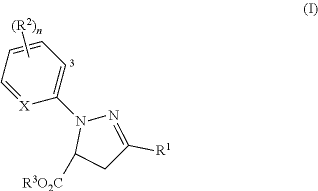
wherein R¹ is halogen;
each R² is independently C₁-C₄ alkyl, C₂-C₄ alkenyl, C₂-C₄ alkynyl, C₃-C₆ cycloalkyl, C₁-C₄ haloalkyl, C₂-C₄ haloalkenyl, C₂-C₄ haloalkynyl, C₃-C₆ halocycloalkyl, halogen, CN, NO₂, C₁-C₄ alkoxy, C₁-C₄ haloalkoxy, C₁-C₄ alkylthio, C₁-C₄ alkylsulfinyl, C₁-C₄ alkylsulfonyl, C₁-C₄ alkylamino, C₂-C₈ dialkylamino, C₃-C₆ cycloalkylamino, C₃-C₆ (alkyl)cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl;
R³ is H or C₁-C₄ alkyl;
X is N or CR⁴;
R⁴ is H or R²; and
n is 0, 1, 2, or 3, with the proviso that when X is CH then n is at least 1;
B) an organic solvent; and
C) an inorganic base;
II) optionally heating the mixture;
III) irradiating the mixture; and
IV) adding an oxidizing agent to the mixture.

2. The method of claim 1 wherein m is 1, 2, or 3.

3. The method of claim 1 wherein R^{s} is Cl or Br.

4. The method of claim 1 wherein R⁶ is independently Cl or Br.

5. The method of claim 4 wherein one R⁶ is at the 3-position.

6. The method of claim 1 wherein R⁷ is C₁-C₄ alkyl.

7. The method of claim 1 wherein Y is N.

8. The method of claim 1 wherein the compound of Formula (II) is ethyl 3-bromo-1-(3-chloro-2-pyridinyl)-1H-pyrazole-5-carboxylate, having the following structure:

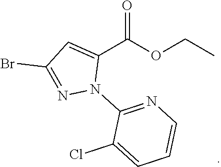 [SMILES: CCOC(=O)c1cc(Br)nn1-c1ncccc1Cl]

9. The method of claim 1, further comprising isolating the compound of Formula (II).

10. The method of claim 1 wherein n is 1, 2, or 3.

11. The method of claim 1 wherein R¹ is Cl or Br.

12. The method of claim 1 wherein R² is independently Cl or Br.

13. The method of claim 12 wherein one R² is at the 3-position.

14. The method of claim 1 wherein R³ is C₁-C₄ alkyl.

15. The method of claim 1 wherein X is N.

16. The method of claim 1 wherein the compound of Formula (I) is ethyl 3-bromo-1-(3-chloropyridin-2-yl)-4,5-dihydro-1H-pyrazole-5-carboxylate, having the following structure:

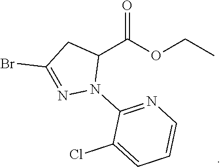 [SMILES: CCOC(=O)C1CC(Br)=NN1c1ncccc1Cl]

17. The method of claim 1 wherein the organic solvent is selected from acetonitrile, N,N-dimethylformamide, dimethylacetamide, chloroform, acetone, propionitrile, chlorobutane, chlorobenzene, tetrachloromethane, dichlorobenzene, dichloromethane, 1,2-dichloroethane, and combinations thereof.

18. The method of claim 1 wherein the inorganic base is selected from sodium bicarbonate, potassium bicarbonate, calcium bicarbonate, sodium carbonate, potassium carbonate, ammonium carbonate, ammonium bicarbonate, trisodium phosphate, tripotassium phosphate, caesium carbonate, triethylamine, pyridine, N-methylimidazole, potassium hydrogen phosphate, sodium hydrogen phosphate, sodium hydroxide, potassium hydroxide, and combinations thereof.

19. The method of claim 1 wherein the inorganic base is in the form of a solid or an aqueous solution.

20. The method of claim 1 wherein the mixture is a two-phase mixture.
